(19) United States
(12) Patent Application Publication
(10) Pub. No.: US 2010/0023114 A1
(43) Pub. Date: Jan. 28, 2010

(54) VALVE DEVICE WITH BIASED LEAFLETS

(75) Inventors: Sean Chambers, Bloomington, IN (US); Ram H. Paul, Bloomington, IN (US)

Correspondence Address: BUCHANAN INTELLECTUAL PROPERTY OFFICE LLC, P.O. BOX 700, PERRYSBURG, OH, 43552-0700, US

(73) Assignee: COOK INCORPORATED, Bloomington, IN (US)

(21) Appl. No.: 12/508,606

(22) Filed: Jul. 24, 2009

Related U.S. Application Data
(60) Provisional application No. 61/083,190, filed on Jul. 24, 2008.

(51) Int. Cl. A61F 2/06

(52) U.S. Cl. 623/1.24

(57) ABSTRACT

Valve devices useful in the treatment of various valve-related disorders and/or conditions are described. A valve device includes a leaflet and a means for biasing the leaflet in an open configuration. A method of making a valve device comprises the steps of determining a desired closing pressure; determining a desired opening pressure, selecting one or more valve leaflets; selecting an appropriate means for biasing the leaflet in an open configuration based on the determined closing and opening pressures; and attaching the one or more valve leaflets to the means for biasing the leaflet in an open configuration.

### CROSS REFERENCE TO RELATED APPLICATION

This application claims priority to U.S. Provisional Application Ser. No. 61/083,190, filed on Jul. 24, 2008. The entire contents of this provisional application are hereby incorporated by reference into this disclosure.

### FIELD

The invention relates generally to the field of implantable medical devices. More particularly, the invention relates to valve devices suitable for regulating fluid flow through a body vessel, and associated methods. Specific embodiments are described that relate to prosthetic venous valves.

### BACKGROUND

Many vessels in animals transport fluids from one bodily location to another. Frequently, fluid flows in a unidirectional manner along the length of the vessel. Varying fluid pressures over time, however, can introduce a reverse flow direction in the vessel. In some vessels, such as mammalian veins, natural valves are positioned along the length of the vessel and act as one-way check valves that open to permit the flow of fluid in the desired direction, and quickly close upon a change in pressure, such as a transition from systole to diastole, to prevent fluid flow in a reverse direction, i.e., retrograde flow.

While natural valves may function for an extended time, some may lose effectiveness, which can lead to physical manifestations and pathology. For example, venous valves are susceptible to becoming insufficient due to one or more of a variety of factors. For example, the vessel wall may stretch over time, affecting the ability of the valve leaflets to close. Furthermore, the leaflets may become damaged, such as by formation of thrombus and scar tissue, which may also affect the ability of the valve leaflets to close. Once valves are damaged, venous valve insufficiency may be present, which can lead to a variety of clinical manifestations, ranging from mild discomfort to non-healing ulcers in the legs and ankles.

Current treatments for venous valve insufficiency include the use of compression stockings that are placed around the leg of a patient in an effort to force the vessel walls radially inward to restore valve function. Surgical techniques are also employed in which valves can be bypassed or replaced with autologous sections of veins with competent valves. Leaflets can also be reconstructed surgically. Ablation of the vessel, such as by application of thermal, radiofrequency, or light energy, is also a common approach to treating superficial vessels with ineffective valves.

Various types intraluminal medical devices that are deployed by minimally invasive techniques have been developed over recent years, including various types of stents, stent grafts, filters, and occluders. To date, however, a prosthetic venous valve deployable in this manner has yet to reach the commercial market despite significant research and development efforts in the field. The prior art includes several examples of valve devices in which a graft or other flexible member is attached to a support structure in a manner that provides a valve function to the device. For example, the graft member can be in the form of a leaflet that is attached to a stent and movable between first and second positions. In a first position, the valve is open and allows fluid flow to proceed through a vessel in a first direction, and in a second position the valve is closed to prevent fluid flow in a second, opposite direction. An example of this type of prosthetic valve is described in commonly owned U.S. Pat. No. 6,508,833, to Pavcnik for a MULTIPLE-SIDED INTRALUMINAL MEDICAL DEVICE, which is hereby incorporated by reference in its entirety. In other examples of prosthetic valves, a tube that terminates in leaflets is attached to one or more support structures to form a valve. The leaflets open to permit fluid flow in a first direction in response to fluid pressure on one side of the leaflets, and close to prevent fluid flow in a second, opposite direction in response to fluid pressure on opposite sides of the leaflets. An example of this configuration is provided in U.S. Pat. No. 6,494,909 to Greenhalgh for AN ENDOVASCULAR VALVE, which is hereby incorporated by reference in its entirety.

Natural valves regulate fluid flow by responding to pressure differentials created by the dynamic fluid environment in the valve area of a body vessel. The leaflets of some natural valves, including natural venous valves, tend to remain in an open configuration absent a change in the surrounding environment, such as a change in one or more of the various fluid pressures exposed to the leaflet. For example, it is believed that the leaflets of the valves remain in an open configuration until a leaflet surface is exposed to sufficient fluid pressure that allows the leaflet to move to a closed position, thereby effecting closure of the valve.

For at least this reason, the inventors believe that prosthetic valves with biased leaflets may lead to viable treatment options for a variety of valve-related disorders and/or conditions, including venous valve insufficiency, cardiac valve disorders, and other situations.

### SUMMARY OF EXEMPLARY EMBODIMENTS

This disclosure describes a variety of valve devices and related methods.

Valve devices for implantation in a body vessel are described.

A valve device according to an exemplary embodiment comprises a valve leaflet movable between first and second positions and means for biasing the leaflet in the first position. The means for biasing the leaflet in the first position is attached to a surface of the leaflet.

A valve device according to another exemplary embodiment comprises a valve leaflet having proximal and distal surfaces and moveable between first and second positions; and a spine attached to the valve leaflet and biased to maintain the valve leaflet in the first position absent application of sufficient force on one of the proximal and distal surfaces. The spine comprises a main body attached to one of the proximal and distal surfaces of the leaflet, an anchor portion adapted to engage a portion of said vessel wall, and a flexible hinge disposed between the anchor portion and the main body.

A valve device according to another exemplary embodiment comprises a valve leaflet having proximal and distal surfaces and moveable between first and second positions; and a spine attached to the valve leaflet and biased to maintain the valve leaflet in the first position absent application of sufficient force on one of the proximal and distal surfaces. The spine comprises a main body attached to one of the proximal and distal surfaces of the leaflet, an anchor portion adapted to engage a portion of said vessel wall, and a flexible hinge disposed between the anchor portion and the main body. The spine is formed of a shape memory material. The main body has a tapered width and has a length that is greater than an expected vessel diameter of a body vessel into which the valve device is intended to be implanted.

Methods of making valve devices are also described.

An exemplary method of making a valve device comprises the steps of determining a desired closing pressure; determining a desired opening pressure; selecting one or more valve leaflets; selecting an appropriate means for biasing at least one of the one or more valve leaflets in an open configuration based on the determined closing and opening pressures; and attaching at least one of the one or more valve leaflets to the means for biasing at least one of the one or more valve leaflets in an open configuration.

Additional understanding can be obtained with review of the detailed description of exemplary embodiments, appearing below, and the appended drawings illustrating exemplary embodiments and methods.

### BRIEF DESCRIPTION OF THE DRAWINGS

FIG. 1 is a perspective view of a valve device according to a first exemplary embodiment.

FIG. 2 is a bottom view of the valve device illustrated in FIG. 1.

FIG. 3 is an elevational view of the spine member of the valve device illustrated in FIG. 1.

FIG. 4 is a frontal view of an alternative spine member.

FIG. 5 is a partial sectional view of a body vessel within which the valve device illustrated in FIG. 1 has been deployed. The valve device is illustrated in an open configuration.

FIG. 6 is a partial sectional view of a body vessel within which the valve device illustrated in FIG. 1 has been deployed. The valve device is illustrated in a closed configuration.

FIG. 7 is a perspective view of a valve device according to a second exemplary embodiment.

FIG. 8 is a partial sectional view of a body vessel within which the valve device illustrated in FIG. 7 has been deployed. The valve device is illustrated in an open configuration.

FIG. 9 is a partial sectional view of a body vessel within which the valve device illustrated in FIG. 7 has been deployed. The valve device is illustrated in a closed configuration.

FIG. 10 is a perspective view of a leaflet suitable for use in a valve device according to a third exemplary embodiment.

FIG. 10A is a magnified view of area I highlighted in FIG. 10.

FIG. 10B is a magnified view of an alternative structure for the valve leaflet illustrated in FIG. 10.

FIG. 11 is a partial sectional view of a body vessel within which a valve device according to the third exemplary embodiment has been deployed. The valve device is illustrated in an open configuration.

FIG. 12 is a partial sectional view of a body vessel within which a valve device according to the third exemplary embodiment has been deployed. The valve device is illustrated in a closed configuration.

FIG. 13 is a perspective view of a valve device according to a fourth exemplary embodiment.

FIG. 14 is a perspective view of a valve device according to a fifth exemplary embodiment.

FIG. 15 is a flowchart illustrating an exemplary method of making a valve device.

FIG. 16 is an end view of a valve device according to an embodiment. The valve device is illustrated in an open configuration.

FIG. 17 is an end view of the valve device illustrated in FIG. 16. The valve device is illustrated in a closed configuration.

### DETAILED DESCRIPTION OF EXEMPLARY EMBODIMENTS

The following detailed description and the appended drawings describe and illustrate various exemplary embodiments of the invention. The description and drawings are exemplary in nature and are provided to enable one skilled in the art to make and use one or more embodiments of the invention. They are not intended to limit the scope of the invention, or its protection, in any manner.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel. None of these terms, or their related forms, require permanent fixation to a portion of a body, such as a body vessel wall or any other portion thereof.

As used herein, the term “deployed” refers to the positioning of a medical device at a location within a body, such as within a body vessel. The term does not refer to any particular delivery device structure or delivery technique.

As used herein, the term “spine” refers to a structure attached to or integrally formed by a leaflet intended for use in a body vessel. The term does not refer to any particular material, dimensions, rigidity, or other characteristics.

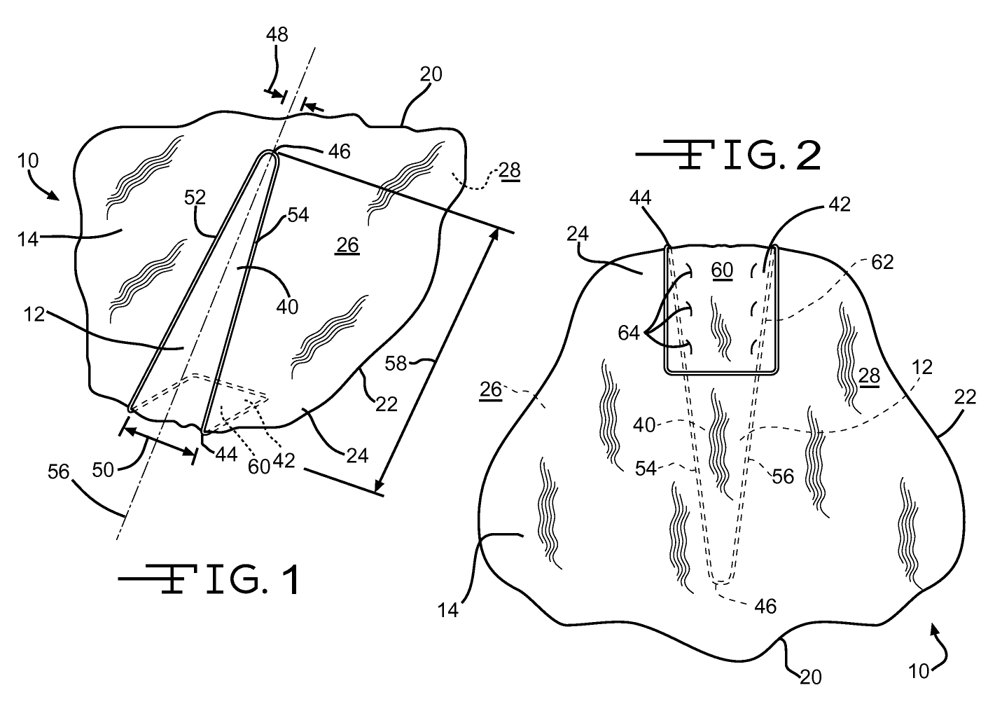

FIGS. 1 through 3, 5 and 6 illustrate a valve device 10 according to a first exemplary embodiment. The valve device 10 is an implantable medical device that comprises a spine 12 attached to a valve leaflet 14. The spine 12 is an example of a suitable means for biasing a valve leaflet in an open configuration.

The leaflet 14 includes first 20 and second 22 edges, a base portion 24, and proximal 26 and distal 28 leaflet surfaces.

A wide variety of materials acceptable for use as the leaflet 14 are known in the art, and any suitable material can be utilized. The material chosen need only be able to perform as described herein, and be biocompatible, or able to be made biocompatible. Examples of suitable materials include flexible materials, natural materials, synthetic materials, and suitable combinations thereof. Examples of suitable natural materials include collagen, extracellular matrix (ECM) materials, such as submucosa, and other bioremodellable materials, such as bovine pericardium. Small intestine submucosa (SIS) is particularly well-suited for use as the graft members 14, 16. Other examples of ECM materials that can be used for the graft member include stomach submucosa, liver basement membrane, urinary bladder submucosa, tissue mucosa, and dura mater. ECMs are particularly well suited materials for use in the graft member, at least because of their abilities to remodel and become incorporated into adjacent tissues. These materials can provide a scaffold onto which cellular in-growth can occur, eventually allowing the material to remodel into a structure of host cells. Other natural materials that can be used include sections of natural animal tissue, including tissue sections, tissue valves, and natural valve leaflets. If used, tissue sections advantageously can be treated according to accepted techniques and/or processes to render the tissue section more appropriate for implantation in a particular animal, such as humans. For example, tissue sections can be fixed according to accepted techniques and/or processes.

Examples of suitable synthetic materials for use in the leaflet 14 include polymeric materials, such as polypropylene, polyurethane, and expanded polytetrafluoroethylene (ePTFE).

The spine 12 includes a main body 40 and an anchor portion 42. A hinge 44 is disposed between the main body 40 and anchor portion 42 and enables the main body 40 to move toward and away from the anchor portion 42 in response to pressure applied to the surfaces 26, 28 of the leaflet 14 when the anchor portion 42 is maintained in a substantially stable position. For example, the hinge 44 enables the main body 40 to move toward and away from the anchor portion 42 in response to fluid pressures when the valve device 10 is implanted in a body vessel and/or in response to manually applied pressures when the valve device 10 is rested on a flat extracorporeal surface, such as a laboratory bench top or other suitable surface.

In this embodiment, the spine 12 is attached to the proximal surface 26 of the leaflet 14. Attaching the spine 12 to the leaflet 14 in this manner is considered advantageous at least because it shields a portion of the proximal surface 26 of the leaflet 14 from fluid flow, which may be desirable in valve devices including leaflets made from materials for which thrombogenecity or other potential results of interaction with fluid is a concern. The attachment between the spine 12 and leaflet 14 can be made in any suitable manner, including via adhesives, attachment members, such as sutures and the like, tissue welding, and other suitable attachment structures, techniques, and/or processes. The specific manner in which the spine 12 and leaflet 14 are attached in a valve device according to a particular embodiment of the invention will depend on various considerations, including the materials used in the spine and the leaflet, and the environment in which the valve device is intended to be disposed during use. Alternatively, the spine can be embedded within the leaflet, such as by being disposed within a pocket defined by the leaflet and adapted to substantially receive the spine.

Also alternatively, the spine can be integrally formed by the leaflet. The leaflet can include a relatively thick portion that integrally forms a spine relative to the remaining, relatively thin portions of the leaflet. For example, a leaflet can be formed of a suitable material, such as a polymeric material, with a relatively thick spine portion molded therein, providing a unitary leaflet/spine apparatus. Other alternatives for integral spines include leaflets with portions having double, triple, and other multi-layer thicknesses as compared to remaining portions of the leaflet having single layer or other thicknesses that are less than that of the spine portion. Other examples include leaflets with portions that have been chemically or otherwise treated to confer a degree of stiffness, rigidity, and/or reduced flexibility onto the treated portion of the leaflet relative to other, non-treated portions of the leaflet. For example, a leaflet formed of a natural material can have an integral spine formed by chemical or other fixation techniques that have been performed on a portion of the leaflet but not on other portions thereof.

The hinge 44 is configured so that the main body 40 is biased in a position that represents an open configuration or position in a body vessel within which the valve device 10 is intended to be implanted. That is, the hinge 44 is configured such that the valve device 10, in a resting state, has a height, hereinafter referred to as the “resting height,” that is less than the expected or actual inner diameter of the vessel in which the valve device is intended to be implanted. FIG. 5 illustrates the relationship between the resting height, represented by dimension indicator 97, and inner vessel diameter, represented by dimension indicator 96, and is described more fully below. As used herein, the term “resting height” refers to a height of a valve device when the valve device is substantially free of external, not atmospheric pressures. The expected diameter can be a measured diameter of an actual vessel into which the device 10 will be implanted, an average or mean diameter determined from measurements made on one or more vessels, a reference value, or a calculation based on one or more reference values. For example, if the valve device 10 is intended to be implanted in a body vessel having a diameter of 1 cm, a valve device can be made with a hinge that confers a resting height on the valve of less than 1 cm.

The inventors have determined that valve devices having a resting height of between about 0.1% and about 75% of the expected or actual inner diameter of the vessel into which they are intended to be implanted are particularly desirable. Valve devices having a resting height of less than about 75% of the expected or actual inner diameter of the vessel into which they are intended to be implanted are considered advantageous. Valve devices having a resting height of less than about 50% of the expected or actual inner diameter of the vessel into which they are intended to be implanted are also considered advantageous. Valve devices having a resting height of less than about 25% of the expected or actual inner diameter of the vessel into which they are intended to be implanted are also considered advantageous. Valve devices having a resting height of less than about 10% of the expected or actual inner diameter of the vessel into which they are intended to be implanted are also considered advantageous.

For prosthetic venous valves, the inventors have determined that resting heights of between about 0.1% and about 75% of the expected or actual inner diameter of the vessel into which they are intended to be implanted are particularly desirable. Prosthetic venous valves having a resting height of less than about 75% of the expected or actual inner diameter of the vessel into which they are intended to be implanted are considered advantageous. Prosthetic venous valves having a resting height of less than about 50% of the expected or actual inner diameter of the vessel into which they are intended to be implanted are also considered advantageous. Prosthetic venous valves having a resting height of less than about 25% of the expected or actual inner diameter of the vessel into which they are intended to be implanted are also considered advantageous. Prosthetic venous valves having a resting height of less than about 10% of the expected or actual inner diameter of the vessel into which they are intended to be implanted are also considered advantageous.

The hinge 44 is also configured such that the bias described above is overcome when the distal leaflet surface 28 has sufficient fluid pressure applied to it. The hinge 44 is advantageously configured such that the threshold fluid pressure that must be present to overcome the bias is a fluid pressure that can be achieved by retrograde fluid flow that occurs in the vessel within which the valve device is intended to be implanted. For example, as described above, natural retrograde flow in the venous system places fluid pressure on distal leaflet surfaces of natural venous valves, causing their closure in normal functioning valves. In venous valve embodiments, the hinge 44 is advantageously configured such that the threshold fluid pressure that must be present to overcome the bias of the spine 12 is within a range of fluid pressures normally produced by retrograde flow that occurs in a vein at the site of a natural venous valve. The inventors have determined that the hinge is advantageously configured such that the threshold fluid pressure that must be present to overcome the bias of the spine 12 is between about 10% and about 250% of the maximum fluid pressure normally produced by retrograde flow that occurs in a vein at the site of a natural venous valve. A threshold pressure between about 25% and about 150% of the maximum fluid pressure normally produced by retrograde flow that occurs in a vein at the site of a natural venous valve is considered more advantageous. A threshold pressure between about 50% and about 100% of the maximum fluid pressure normally produced by retrograde flow that occurs in a vein at the site of a natural venous valve is considered more advantageous. A threshold pressure between about 75% and about 100% of the maximum fluid pressure normally produced by retrograde flow that occurs in a vein at the site of a natural venous valve is considered more advantageous. A threshold pressure of about 100% of the maximum fluid pressure normally produced by retrograde flow that occurs in a vein at the site of a natural venous valve is considered particularly advantageous. It is noted that, in vivo, a deceleration of antegrade flow may also be partly or wholly responsible for overcoming the bias of the spine.

It is believed that, for prosthetic venous valves adapted and intended for implantation in animals, such as humans, a hinge configured such that the bias of the spine is overcome when the leaflet surface is exposed to fluid pressures greater than about 10 mmHg and less than about 30 mmHg is suitable. The low end of this range represents a typical maximum fluid pressure in a human femoral vein while lying down, and the high end represents a typical maximum fluid pressure in a human femoral vein while standing. Other hinges configured according to other pressure values and ranges can be used, of course. The skilled artisan will be able to configure an appropriate hinge for a particular valve device according to an embodiment of the invention based on various considerations, including typical, expected, or actual fluid pressures present in the body vessel in which the valve device is intended to be implanted.

The hinge 44 can have any suitable structure that achieves the desired function as described herein. As indicated above, the hinge 44 enables the main body 40 to move toward and away from the anchor portion 42 when the anchor portion 42 is maintained in a substantially stable position. The hinge 44 can comprise any suitable structure that achieves this desired result. For example, the hinge 44 can comprise a simple bend in the material forming the spine 12. FIG. 3 illustrates a suitable structure for the hinge 44. In this embodiment, the hinge 44 comprises a bend 70 disposed between the main body 40 and the anchor portion 42. The hinge 44 includes a section of material 72 that has a thickness 76 that is less than a thickness 74 of the main body 40. This reduced thickness provides desirable flexibility for the spine 12 by allowing the main body 40 to move more freely than if a constant thickness was employed throughout the entire spine 12. As illustrated in FIG. 3, the section 72 having the reduced thickness 76 is advantageously positioned immediately adjacent the bend 70 on the main body 40 side of the hinge 44. This configuration provides a substantially uniform thickness to the anchor portion 42 (without factoring in any barbs 64 associated with the anchor portion 42). It is noted that, while the illustrated main body 40 has a constant thickness over its length, a varying thickness can be used for this portion of the spine as well. It is believed that a varying thickness for the main body 40, with or without the reduced thickness section 72, can enhance flexibility and/or responsiveness of the spine 12 when the valve device 10 is implanted in a body vessel.

FIG. 4 illustrates an alternate spine 12′. The spine 12′ is an example of a suitable means for biasing the leaflet 14 in an open configuration. In this embodiment, the spine 12′ comprises a main body 40′, an anchor portion 42′, and a hinge 44′ disposed between the main body 40′ and the anchor portion 42′. In contrast to the spine 12 illustrated in FIG. 3, the spine 12′ includes a tip 46′ having a width 48′ that is substantially the same as the width 50′ of the main body 40′ adjacent the hinge 44′. This configuration may be advantageous, despite the additional material requirements, in valve devices that require additional support and/or attachment surface area between the spine and the leaflet.

Also in contrast to the spine 12 illustrated in FIG. 3, the hinge 44′ in the alternate spine 44′ illustrated in FIG. 4 has a substantially uniform thickness. The spine 12′ includes, however, a series of openings 55, 57 that reduce the overall amount of material in the hinge 44′ and, as a result, contribute the to desired flexibility of the spine 12′. As illustrated in FIG. 4, both enclosed 55 and partially enclosed 57 openings can be included. Alternatively, one type of opening can be used exclusively. Furthermore, any suitable number of openings can be used in a particular spine; the exact number and type chosen will depend on various considerations, including the desired flexibility of the spine.

The openings 55, 57 can also provide an opening through which a controlled amount of retrograde flow can be allowed to pass when the valve device is in a closed configuration. The leaflet should be attached in a manner that allows one or more of the openings 55, 57 to provide fluid communication between portions of the body vessel in which the device is implanted that lie on opposite sides of the leaflet.

As best illustrated in FIG. 1, the main body 40 of the spine 12 includes a tip 46 opposite the hinge 44. The tip 46 advantageously has rounded edges, which are believed to reduce wear on the leaflet 14 during use of the valve device 10. A first width 48, measured at the tip 46, is less than a second width 50, measured at the hinge 44. This provides a tapered width to the main body 40 that transitions from the first width 48 to the second width 50. This tapered width is expected to be advantageous in that it allows for use of less material in the spine 12 while retaining the desired function. The main body 40 has first 52 and second 54 edges that are equidistantly spaced from a hypothetical midline 56. A length 58 of the main body 40 extends from the hinge 44 to the tip 46.

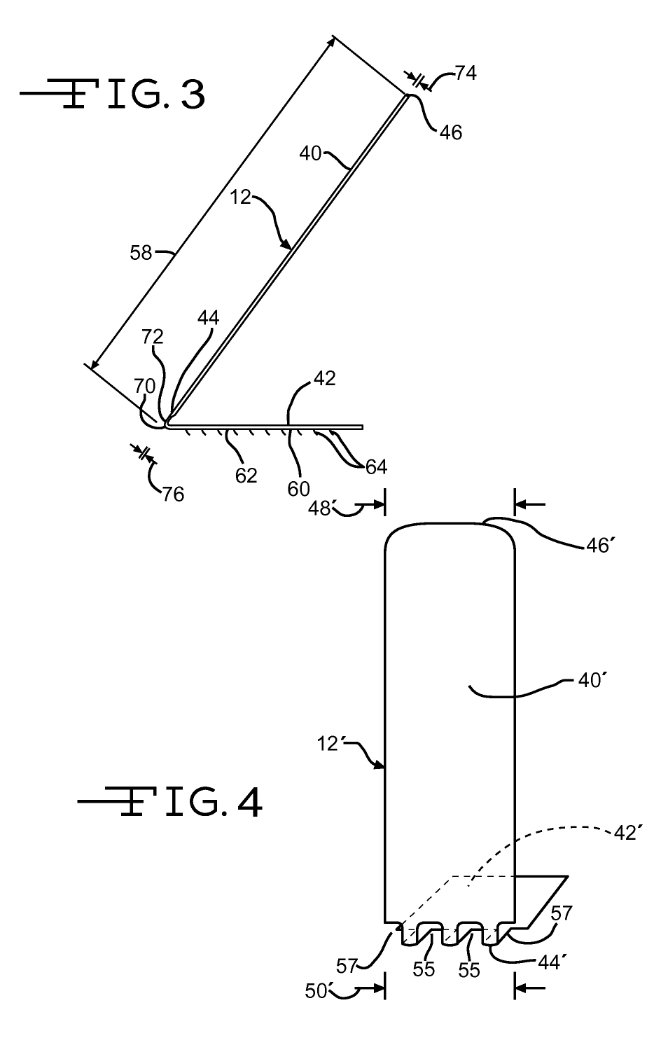

The anchor portion 60 comprises a substantially flat section positioned at an angle to the main body 40. As best illustrated in FIG. 2, an under surface 62 of the anchor portion 60, which is disposed opposite the leaflet 14, provides a contact area 64 adapted to contact body tissue at a point of implantation in a body, such as a portion of a vessel wall. The contact area 64 advantageously includes one or more barbs 64, which individually comprise a projection from the surface 62 that is adapted to pierce through a desired thickness of the tissue expected at the desired point of implantation. A plurality of barbs 64 disposed in a substantially regular pattern on the surface 62 facilitates the formation of a desirable attachment between the contact area 64 and the body tissue at the point of implantation. It is understood that the anchor portion 60 can comprise a portion of any suitable structure that provides the spine 12, such as a support frame, stent, or other intraluminal suitable device.

The spine 12 can be formed of any suitable material. Plastics, other polymeric materials, metals, and other biocompatible materials can be used. The material need only be able to allow the spine 12 to perform as described herein. Advantageously, the spine 12 is made of a flexible plastic material or a shape memory material, such as nitinol or other suitable shape memory alloys. Shape memory polymers, such as those described in U.S. Pat. No. 6,720,402 to Langer for SHAPE MEMORY POLYMERS, the entire contents of which are hereby incorporated into this disclosure, can also be used. Nitinol is considered particularly advantageous at least because of its established biocompatibility, ready availability, and well-characterized shape memory properties and behavior. Also, while the spine 12 is illustrated as being made of a single material, multiple materials can be used. For example, the main body and anchor portions can be made of a relatively rigid material, and the hinge can be made of a relatively flexible material that is joined or otherwise attached to the main body and anchor portions. Also, as noted above, the spine can alternatively be integrally formed with the leaflet. Also, the spine can have any suitable configuration and need only be able to perform as described herein. Indeed, a simple wire structure may be used as an alternative spine structure.

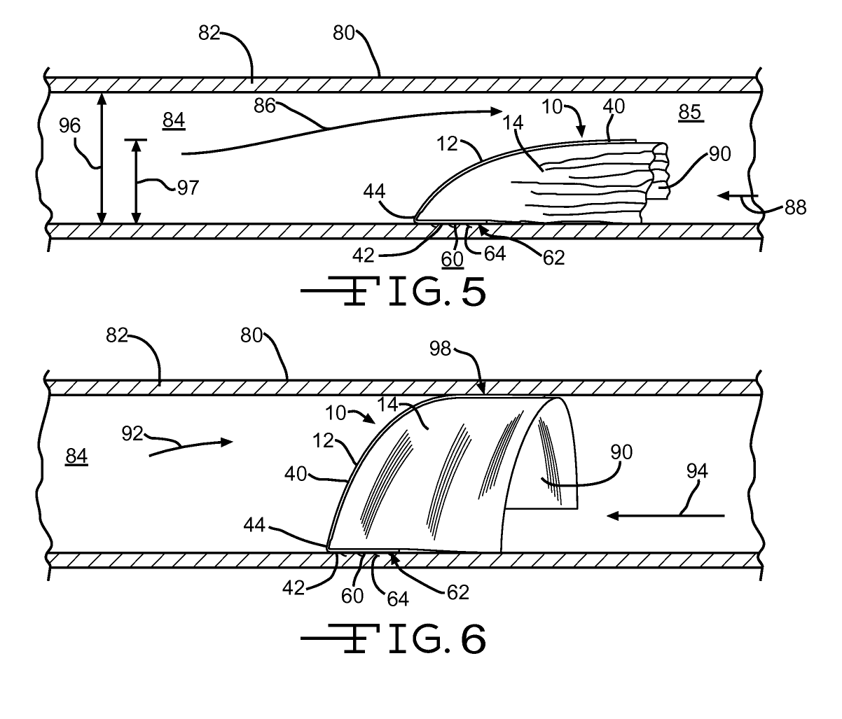

FIGS. 5 and 6 illustrate the valve device 10 deployed within a body vessel 80. FIG. 5 illustrates the valve device 10 in an open configuration; FIG. 6 illustrates the valve device 10 in a closed configuration.

The body vessel 80 includes vessel wall 82 that defines lumen 84. The body vessel 80 can be any suitable body vessel through which a user desires to regulate fluid flow. Exemplary vessels include veins, arteries, coronary vessels, and ducts that transport one or more body fluids from one location to another. Valve devices according to particular embodiments are particularly well-suited for use as prosthetic venous valves for deployment in a vein of an animal, such as a human, for the purpose of regulating the flow of blood therethrough.

The contact area 62 of the anchor portion 42 is in contact with the vessel wall such that barbs 64 pierce through at least a portion of the thickness of the vessel wall 82. As such, the barbs 64 maintain the axial location of the valve device 10 in the body vessel 80 as it transitions between open and closed configurations.

The body vessel 80 has an internal vessel diameter 96. As described above, the resting height 97 of the valve device 10 is less than the vessel diameter 96. The resting height 97 is defined by the extent of the bias of the spine 12 and represents the maximum height of the valve device 10 when in the open configuration. Also, as best illustrated in FIG. 6, the length of the main body 40 of the spine 12 (note: the length is represented by reference number 58 in FIG. 1; it is not referenced in FIG. 6) advantageously is greater than the vessel diameter 96. This configuration helps to ensure that the valve device 10 does not evert when moving into the closed configuration. The length 58 of the main body 40 is advantageously between about 100% and about 250% of the vessel diameter 96. More advantageously, the length 58 of the main body 40 is between about 100% and about 150% of the vessel diameter 96. More advantageously, the length 58 of the main body 40 is between about 100% and about 125% of the vessel diameter 96. More advantageously, the length 58 of the main body 40 is between about 100% and about 110% of the vessel diameter 96. More advantageously, the length 58 of the main body 40 is between about 100% and about 105% of the vessel diameter 96.

In FIG. 5, the valve device 10 is illustrated in an open configuration. As described above, the spine 12 is biased to substantially maintain this configuration absent sufficient fluid pressure on the distal leaflet surface 28 to overcome the bias. Thus, absent sufficient pressure on the distal leaflet surface 28, the leaflet 14 maintains a substantially collapsed configuration that provides a passageway 85 through which fluid flow can pass, crossing the valve device 10 and proceeding distally in the body vessel 80. Accommodating for the material of the valve device 10, the passageway 85 is a portion of the cross-sectional area of the vessel lumen 84.

With reference to FIGS. 5 and 6, in vivo functioning of the valve device 10 will now be described. When the valve device 10 is in its biased open configuration, illustrated in FIG. 5, antegrade fluid flow, represented by arrow 86, flows across the valve device 10, rushing past proximal leaflet surface 26 and spine 12. Initially, a relatively small amount of retrograde flow, which proceeds in the opposite direction within the body vessel 80 and which is represented by arrow 88, presents fluid to the distal leaflet surface 28. This retrograde flow 88 gradually fills a valve pocket 90 formed by the valve device 10. Over a period of time, the retrograde flow 88 produces sufficient fluid pressure within the valve pocket 90 to overcome the biased position of the hinge 44 on the spine 12. Once the necessary fluid pressure is present, the bias of the spine 12 is overcome and the main body 40 moves away from the anchor portion 42, eventually achieving the closed configuration illustrated in FIG. 6. It is noted, as above, that, in vivo, a deceleration of antegrade flow may actually account in whole or in part for overcoming the bias of the spine 12.

As best illustrated in FIG. 6, the retrograde flow 94 continues to fill the valve pocket 90 with fluid, ultimately overcoming the elastic recoil of the spine 12, forcing the leaflet 14 to unfurl and form substantially circumferential contact with the vessel wall 82. While the valve device 10 is in this closed configuration, antegrade flow, illustrated by arrow 92 in FIG. 6, continues to carry fluid to the valve device 10 but distal passage of the fluid is substantially blocked by the leaflet 14 being held in the closed configuration. Ultimately, the elastic recoil of the spine 12, in combination with the antegrade flow 92, produces sufficient pressure on the proximal surface 26 of the leaflet 14 to overcome the fluid pressure exerted on the distal surface 28 of the leaflet 14 in the valve pocket 90. Once a threshold is achieved, the main body 40 of the spine 12 moves toward the anchor portion 42, eventually returning to the open configuration illustrated in FIG. 5. At this point, the bias of the spine 12 is restored and the cycle based on pressure differentials is able to repeat.

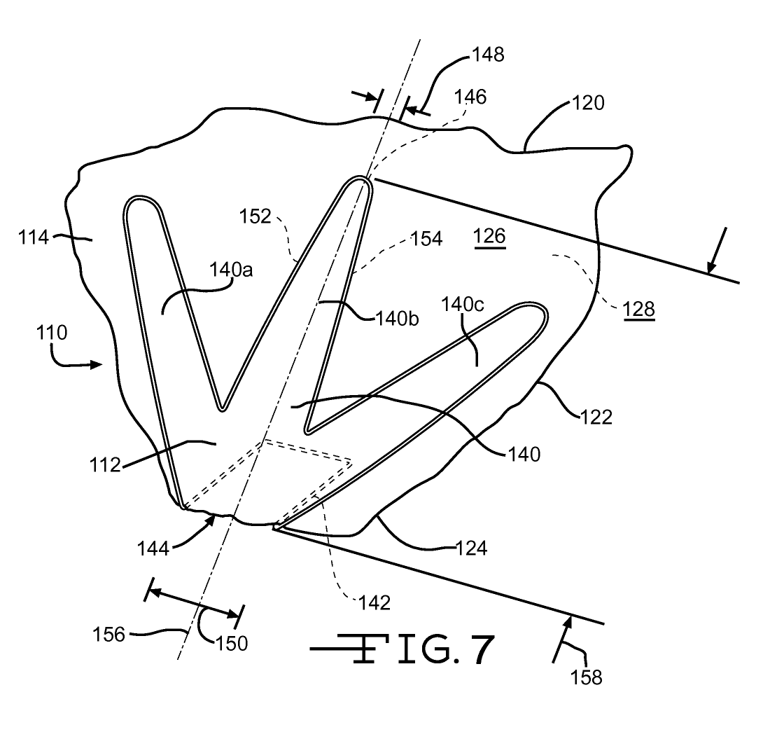

FIGS. 7 through 9 illustrate a valve device 110 according to another exemplary embodiment. The valve device 110 according to this embodiment is similar to the device 10 according to the embodiment illustrated in FIGS. 1 through 3, 5 and 6, and described above, except as detailed below. Thus, the valve device 110 includes a spine 112 attached to a leaflet 114. The leaflet 114 has a first edge 120, a second edge 122, a base 124, and proximal 126 and distal 128 leaflet surfaces. The spine 112 is an example of a suitable means for biasing the leaflet 114 in an open configuration. The spine 112 includes a main body 140, an anchor portion 142, and a hinge 144 disposed between the main body 140 and the anchor portion 142. The hinge 144 provides flexibility that allows the main body 140 to move toward and away from the anchor portion 142 and is biased in an open configuration as described above.

In this embodiment, the main body 140 includes first 140a, second 140b, and third 140c body extensions. Each of the extensions includes a tip 146 and first 152 and second 154 edges that define a taper from a first width 148 to a larger second width 150. Each of the body extensions 140a, 140b, 140c terminate in a common section adjacent the hinge 144. At least one of the body extensions 140a, 140b, 140c, has a length 158, measured from the tip 146 to the hinge 144, that is greater than an expected vessel diameter for a body vessel in which the valve device 110 is intended to be implanted. It is considered advantageous for all of the body extensions 140a, 140b, 140c to have such a length at least because this enhances the ability of the spine 112 to prevent eversion of the leaflet 114 during use of the valve device 110.

The spine 112 in this embodiment is attached to the distal leaflet surface 128. Attaching the spine 112 to the leaflet 114 in this manner is considered advantageous at least because it substantially excludes the attachment between the spine 112 and leaflet 114 from antegrade fluid flow and also affords some protection to the vessel wall from direct contact with the spine 112. Such contact might be undesirable in some embodiments, e.g., valve device that include a spine main body made with of rigid or potentially irritating materials, and/or in some vessels, e.g., vessels that have weakened vessel walls.

As best illustrated in FIG. 7, the inclusion of multiple body extensions 140a, 140b, 140c provides additional contact area between the spine 112 and the leaflet 114 than that provided in the embodiment illustrated in FIG. 1. This is expected to be advantageous at least because the additional contact area provides for a more rugged attachment between the spine 112 and the leaflet 114 and provides additional structural elements that can aid in the prevention of eversion of the valve device 110 during transition from the open configuration to the closed configuration.

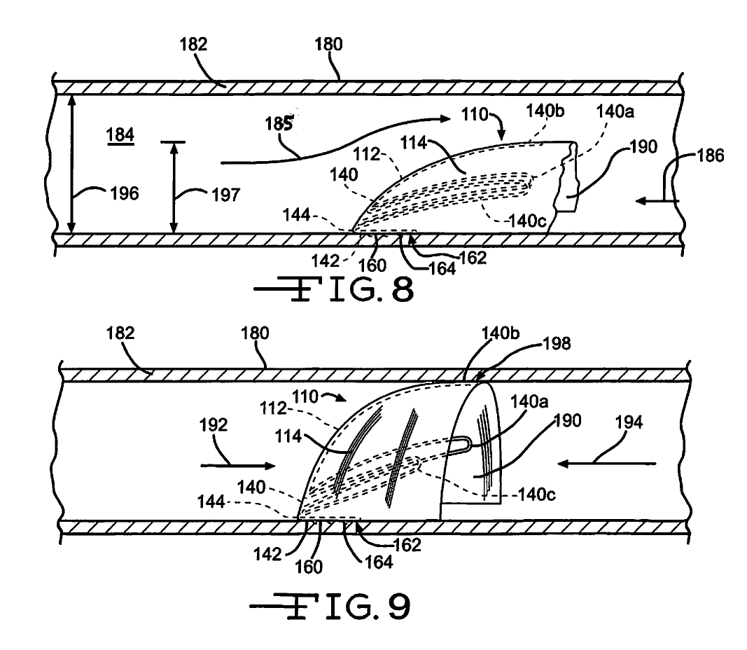

FIGS. 8 and 9 illustrate the valve device 110 positioned in a lumen 184 of a body vessel 180. FIG. 8 illustrates the valve device 110 in an open configuration; FIG. 9 illustrates the valve device 110 in a closed configuration.

The valve device 110 is maintained in the vessel 180 by the interaction between the barbs 164 and the vessel wall 182. In the open configuration, antegrade flow, represented by arrow 185, passes past the valve device 110 while retrograde flow, represented by arrow 186, flows into valve pocket 190 defined by leaflet 114. Fluid accumulates in the valve pocket 190 as retrograde flow continues, eventually presenting sufficient fluid pressure on the distal leaflet surface to overcome the bias of the spine 112. Once this pressure is achieved, the valve device 110 takes on the closed configuration, illustrated in FIG. 6. In this configuration, the leaflet 114 has unfurled and formed a substantially circumferential contact with the vessel wall 182. Additional retrograde flow, represented by arrow 194, continues to force the leaflet 114 into contact with the vessel wall 182 while antegrade flow, represented by arrow 192 presents fluid pressure to the proximal surface 126 of the leaflet 114. Eventually, the fluid pressure presented by the antegrade flow 192, in combination with the elastic recoil of the spine 112, is sufficient to overcome the pressure presented by the retrograde flow 194 and the valve device 110 is returned to an open configuration as the spine 112 is restored to its biased open configuration.

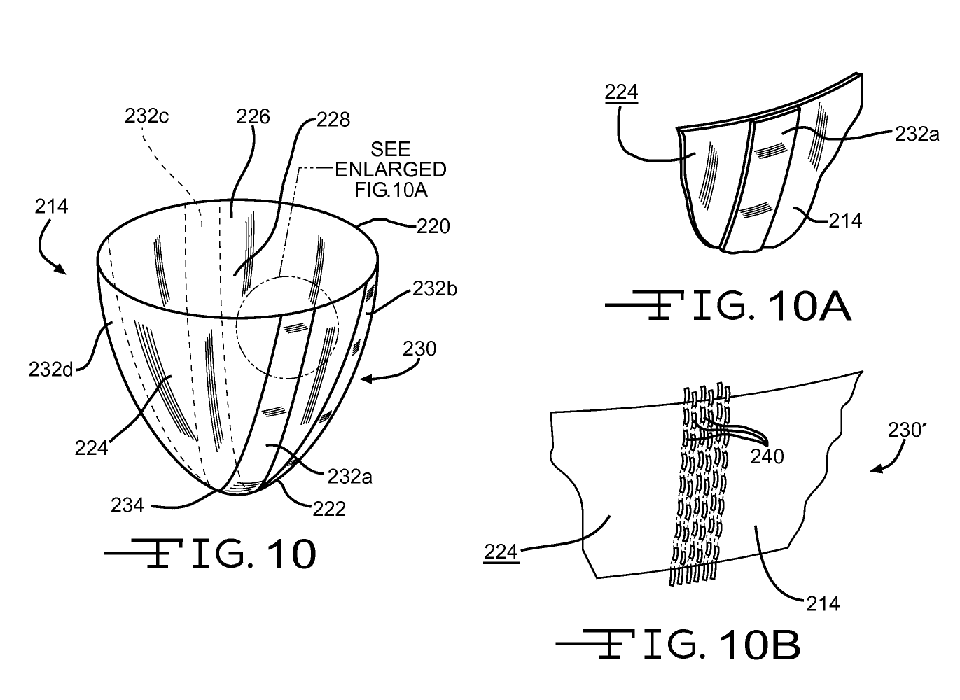

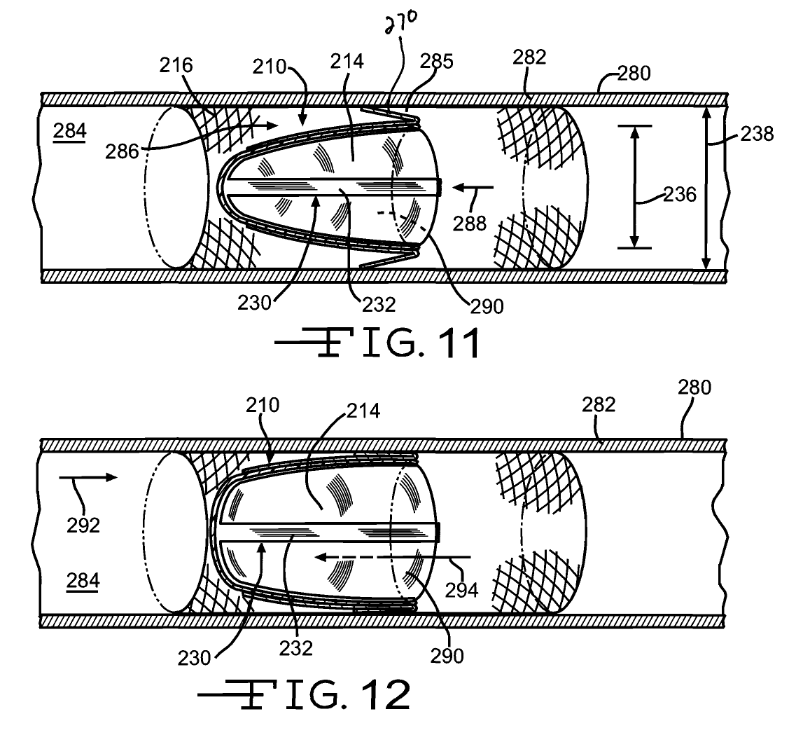

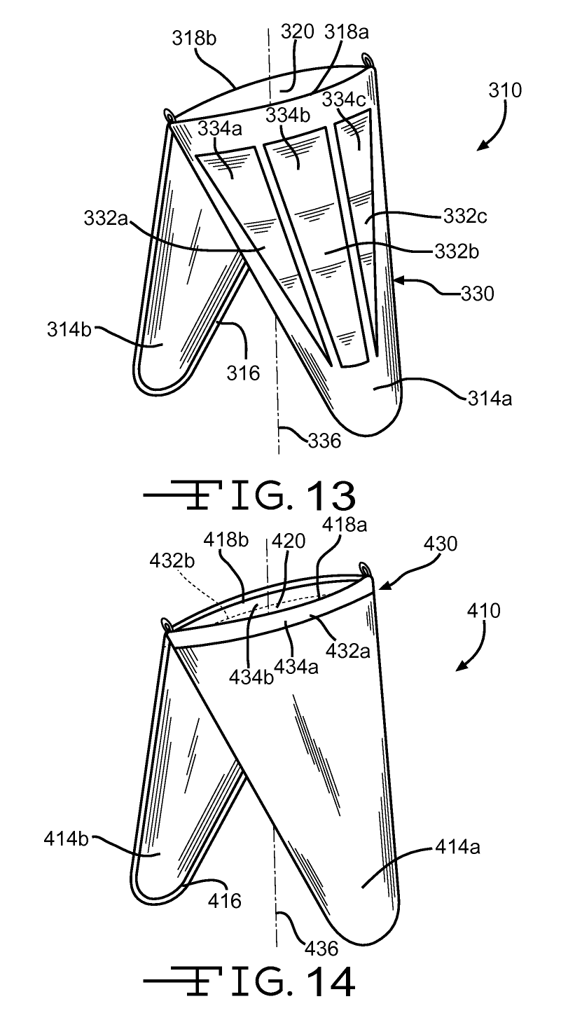

FIGS. 10 through 12 illustrate a valve device 210 according to another exemplary embodiment. For purposes of simplifying illustration and description of this embodiment, FIG. 10 illustrates a leaflet 214 and associated means for biasing 230 the leaflet 214 in an open configuration without the associated support frame, which is illustrated in FIGS. 11 and 12.

In this embodiment, the valve device 210 comprises a leaflet 214 attached to a support frame 270. A means for biasing 230 the leaflet 214 in an open configuration is disposed on the leaflet 214 and comprises one or more strips 232 of shape memory material. As best illustrated in FIG. 10, four strips 232a, 232b, 232c, 232d that originate from a common base 234 provide a suitable means for biasing 230 the leaflet 214 in an open configuration. It is understood, though, that any suitable number of strips can be used and the specific number chosen for a valve device according to a particular embodiment will depend on several considerations, including the size of valve leaflet and the material used in the leaflet. Also, no matter the number of strips used, the strips can be placed on the leaflet in any suitable configuration. As illustrated in FIG. 10, a configuration in which the strips are positioned equidistantly around the circumference of a substantially conical leaflet 214 is considered advantageous at least because such positioning facilitates a uniform opening and closing of the leaflet 214 during use of the valve device 210. Non-equidistant positioning is considered suitable, though, and might be advantageous in particular embodiments. Furthermore, the inclusion of a common base is considered optional—multiple strips that overlap or lack contact altogether can also be used. Also, while the strips 232a, 232b, 232c, 232d are positioned on an external surface 224 of the leaflet 214, strips can also be positioned on an internal surface 226 of the leaflet 214. Such positioning might be advantageous in embodiments in which it is desired to avoid contact between the strips and a vessel wall, for example. Similarly, strips can also be wholly or partially embedded within the material of the leaflet 214. Such positioning might be advantageous in embodiments in which it is desired to avoid contact between the strips and fluid flowing through the body vessel.

FIG. 10A is a magnified view of the area highlighted in FIG. 10; it illustrates the detail of the attachment of the strip 232a to the leaflet 214. In this embodiment, the strip 232a is attached directly to the external surface 224 of the leaflet 214. The attachment can be formed in any suitable manner and using any means for attaching that is suitable for the materials used in the strip 232a and leaflet 214. Examples of suitable means for attaching include adhesives, attachment members, such as sutures, clips, and the like, and tissue welds. The inventors have determined that conventional biocompatible adhesives are suitable for use in valve devices in which the strip comprises nitinol and the leaflet comprises a synthetic material, such as a polymeric material. The inventors have also determined that sutures and other attachment members, such as clips, are suitable for used in valve devices in which the strip comprises nitinol and the leaflet comprises a natural material, such as a tissue section, an ECM, or another natural material. In these embodiments, the strips are advantageously modified to include holes or other suitable structure that facilitates the use of the attachment members.

FIG. 10B illustrates an alternate strip 230′ attached to the leaflet 214. In this embodiment, the strip 230′ comprises a series of filaments 240 that are woven through at least a portion of the thickness of the leaflet 214. Advantageously, the filaments 240 are repeatedly passed through the entire thickness of the leaflet 240. Advantageously, the strip 230′ comprises a plurality of filaments 240, although any suitable number of filaments can be used, including a single filament. For some materials, it may be desirable to pass the filaments through only a portion of the thickness of the leaflet 214, such as thrombogenic and other materials that are desirably shielded from blood and/or other fluids to which a valve device may be exposed in use.

The leaflet 214 in this embodiment is substantially conical in shape, having an edge 220, a base 222, an external surface 224, and an internal surface 226. The leaflet 214 defines an internal valve pocket 228 that, as described in more detail below, opens and closes in response to pressure changes in a body vessel.

With reference to FIGS. 11 and 12, the functioning of the valve device 210 will now be described. FIGS. 11 and 12 illustrate the valve device 210 deployed within a body vessel 280. FIG. 11 illustrates the valve device 210 in an open configuration; FIG. 12 illustrates the valve device 210 in a closed configuration.

The leaflet 214 is attached to support frame 216. Any suitable support frame formed of any suitable material can be used, and a wide variety are known in the art. Both self-expandable and balloon expandable frames can be used, as can other types of frames. The only requirement is that the frame 216 must be able to support the leaflet 216 in a manner that allows it to move between open and closed configurations as described herein.

In the open configuration, the leaflet 214 is circumferentially spaced from the vessel wall 282 to form a circumferential passageway 285 through which antegrade flow, represented by arrow 286 in FIG. 11, can pass. In this configuration, the means for biasing 230 the leaflet 214 maintain the passageway 285 open by keeping the leaflet diameter 236 less than the vessel diameter 238. Gradually, as antegrade flow 286 proceeds past the valve device 210, retrograde flow, represented by arrow 288, flows into the valve pocket 290. Over a period of time, the retrograde flow 288 collects in the pocket and presents fluid pressure to the internal surface of the valve leaflet 214. Once a threshold pressure is achieved, the bias of the means for biasing 230 the leaflet in an open configuration is overcome, and the diameter of the leaflet 214 increases to ultimately achieve the closed configuration illustrated in FIG. 12. In this configuration, the passageway 285 has substantially been closed, substantially preventing antegrade fluid flow, represented by arrow 292 in FIG. 12, from flowing past the valve device 210. Over time, pressure created by antegrade flow 292 exceeds the pressure created by the fluid in the valve pocket 290, represented by arrow 294 in FIG. 12. Once this pressure differential is achieved, the bias of the means for biasing 230 the leaflet 214 in an open configuration is restored, which returns the valve device 210 to the open configuration illustrated in FIG. 11.

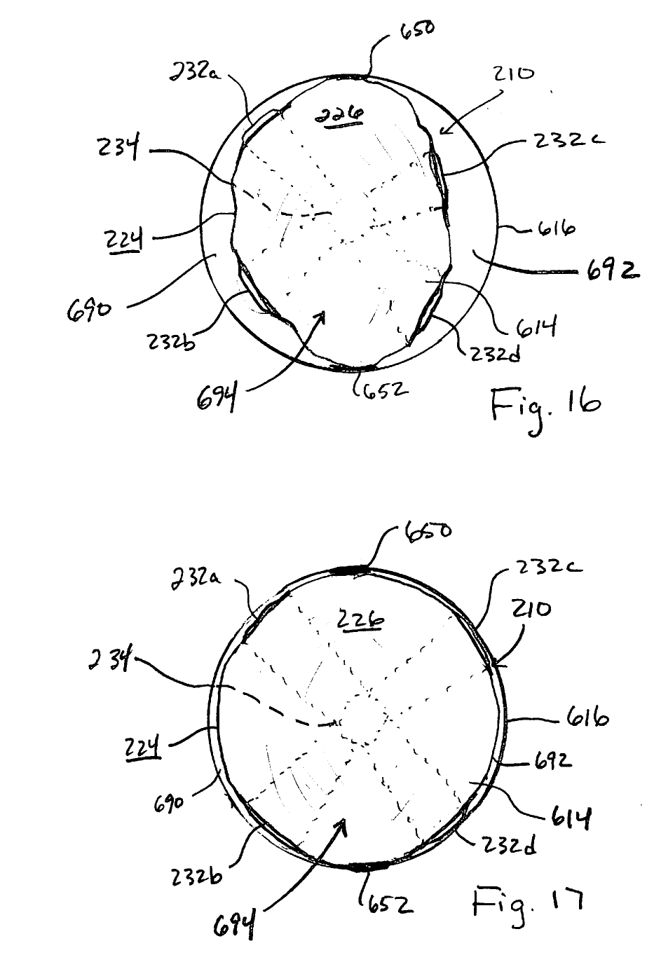

FIGS. 16 and 17 illustrate an alternative structure for using the valve device 210 of FIG. 10 with a support frame. In this embodiment, the leaflet 214 is attached directly to the support frame 616 at first 650 and second 652 attachment points. The attachment points 650, 652 are disposed substantially on the circumference of the support frame 616 and substantially opposite each other. This arrangement ensures that the attachment points 650, 652 are disposed directly adjacent an inner wall of a body vessel in which the device 210 is implanted. The four strips 232a, 232b, 232c, 232d are disposed on the external surface 224 of the leaflet 214 and originate from a common base 234.

The support frame can comprise any suitable support frame, including self-expandable and non-expandable support frames, support frames that require an input of outwardly-directed force to achieve expansion, such as balloon-expandable stents, and any other suitable structure to which the valve device can be attached as described herein. Skilled artisans will be able to select an appropriate support frame for inclusion in a device according to a particular embodiment based on various considerations, including the nature of the leaflet and the vessel into which the device is intended to be implanted.

A support frame that provides specific structure for the attachment points 650, 652 on substantially opposing sides of the support frame 616 are considered particularly advantageous at least because this arrangement facilitates the opening and closing of the valve device 210, as described in more detail below. Examples of suitable support frames include those described in U.S. Pat. No. 7,544,205 to Flagle et al. for INTRALUMINAL SUPPORT FRAME AND MEDICAL DEVICES INCLUDING THE SUPPORT FRAME, the entire contents of which is hereby incorporated into this disclosure for the purpose of describing suitable support frames and other components for use in devices in accordance with the disclosure.

Support frames that provides additional structure are also considered advantageous. For example, a support frame that provides a sinus-defining structure can facilitate the opening and closing of the valve device 210, as described below. Thus, additional examples of suitable support frames include those described in United States Patent Application Publication no. 20090132037 to Hoffman et al. for VALVE FRAME, United States Patent Application Publication no. 20070260327 to Case et al. for ARTIFICAL VALVE PROSTHESIS WITH IMPROVED FLOW DYNAMICS, and United States Patent Application Publication no. 20090105813 to Chambers et al. for IMPLANTABLE VALVE DEVICE, the entire contents of each of which are hereby incorporated by reference into this disclosure for the purpose of describing suitable support frames and other components for use in devices in accordance with the disclosure.

No matter the structure used for the support frame 616, any suitable means for attaching a leaflet to a support frame can be used to form attachment points 650, 652. Skilled artisans will be able to select a suitable structure to use as the means for attaching in a device according to a particular embodiment based on various considerations, including the nature of the leaflet 214 and support frame 616, and the nature of the environment into which the device is intended to be implanted. Examples of suitable structures for the means for attaching include sutures, clips, adhesives, weld joints, hooks, barbs, and the like.

FIG. 16 illustrates the valve device 210 in an open configuration; FIG. 17 illustrates the device 210 in a closed configuration. In the open configuration, the strips 232a, 232b, 232c, 232d pull the leaflet 214 inward, creating two substantially semi-circular openings 690, 692 through which fluid can flow. The valve device 210 eventually transitions to the closed configuration in response to changing fluid pressures or other factors. In the closed configuration, the bias of the strips 232a, 232b, 232c, 232d is overcome and fluid that has collected in the pocket 694 exerts pressure on the internal surface 226. As a result, leaflet 214 is forced outward, substantially closing the openings 690, 692, which substantially prevents fluid flow across the valve device 210. Ultimately, the valve device 210 will revert back to the open configuration, re-forming openings 690, 692 and allowing fluid flow across the valve device 210.

FIG. 13 illustrates a valve device 310 according to another exemplary embodiment. In this embodiment, the valve device 310 comprises first 314a and second 314b valve leaflets attached to a support frame 316. The first leaflet 314a has a first free edge 318a and the second leaflet 314b has a second free edge 318b. The free edges 318a, 318b are not attached to the support frame 316 and cooperatively define a valve orifice 320 that opens and closes in response to changing fluid pressures within a body vessel in which the valve device 310 is implanted. This alternation between open and closed configurations regulates fluid flow through the body vessel. The valve device 310 is a variation of the valve device described in U.S. Pat. No. 7,402,171 to Osborne et. al. for PROSTHETIC VALVE THAT PERMITS RETROGRADE FLOW, the entire contents of which are incorporated by reference into this disclosure for the purpose of describing portions of the valve device 310 according to this exemplary embodiment.

A means for biasing 330 the leaflets 314a, 314b in an open configuration is associated with the leaflets 314a, 314b. In this embodiment, the means for biasing 330 comprises strips 332a, 332b, 332c of a suitable shape memory material disposed on a surface of the leaflets 314a, 314b. The strips 332a, 332b, 332c are trained, in a manner appropriate for the particular shape memory material used in the strips 332a, 332b, 332c to deflect a first end 334a, 334b, 334c outward relative to a longitudinal axis 336 of the valve device 310 when the strips 332a, 332b, 332c are exposed to a body temperature of the animal into which the valve device is intended to be implanted. The first ends 334a, 334b, 334c are advantageously disposed adjacent or substantially adjacent the one or more of the free edges 318a, 318b of the leaflets 314a, 314b. This training of the shape memory strips 332a, 332b, 332c ensures that the valve orifice 320 is biased open and closes only when fluid pressure presented to the leaflets 314a, 314b is able to overcome the tendency of the strips 332a, 332b, 332c to maintain their deflected position. Note that while strips 332a, 332b, 332c are illustrated as having relatively sharp corners, rounded or other corners may be desirable, such as to avoid friction, chafing, tearing, or other potential damage to leaflets 314a, 314b.

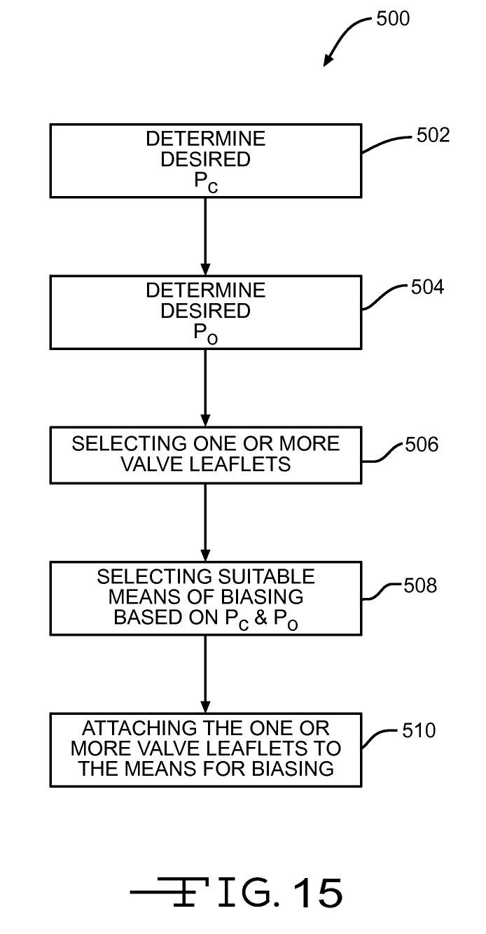

FIG. 14 illustrates a valve device 410 according to another exemplary embodiment. The valve device 410 according to this embodiment is similar to the device 310 according to the embodiment illustrated in FIG. 13 and described above, except as detailed below. Thus, the valve device 410 comprises first 414a and second 414b valve leaflets attached to a support frame 416. The first leaflet 414a has a first free edge 418a and the second leaflet 414b has a second free edge 418b. The free edges 418a, 418b are not attached to the support frame 416 and cooperatively define a valve orifice 420 that opens and closes in response to changing fluid pressures within a body vessel in which the valve device 410 is implanted. The valve device 410 functions in a similar manner as the device 310 described above. This alternation between open and closed configurations regulates fluid flow through the body vessel.

A means for biasing 430 the leaflets 414a, 414b in an open configuration is associated with the leaflets 414a, 414b. In this embodiment, the means for biasing 430 comprises strips 432a, 432b of a suitable shape memory material disposed on the free edges 418a, 418b of the leaflets 414a, 414b. The strips 432a, 432b are trained, in a manner appropriate for the particular shape memory material used in the strips 432a, 432b to deflect an interior point along their respective length, such as midpoints 434a, 434b outward relative to a longitudinal axis 436 of the valve device 410 when the strips 432a, 432b are exposed to a body temperature of the animal into which the valve device is intended to be implanted. This training of the shape memory strips 432a, 432b ensures that the leaflets 414a, 414b are biased in an open configuration. That is, the valve orifice 420 is biased open and closes only when fluid pressure presented to the leaflets 414a, 414b is able to overcome the tendency of the strips 432a, 432b to maintain the deflected position.

As illustrated in FIG. 14, the strips 432a, 432b are advantageously disposed on an external surface of the leaflets 414a, 414b. This positioning is considered advantageous at least because it avoids positioning additional bulk within the valve orifice 420, which could impact fluid flow through the valve device 410. Alternative positioning, such as on the internal surfaces of the leaflets, wholly or partially embedded within the material of the leaflets, or a combination of different positioning of the strips 432a, 432b relative to the leaflets 414a, 414b can be utilized and may be desirable in valve devices according to particular embodiments. Also alternatively, as described above, the strips can be integrally formed by the leaflet, such as by a relatively thick and/or chemically or otherwise treated portion of the leaflet.

It is understood that various structures, configurations and materials can be used for the support frames and leaflets in embodiments that include these elements. Indeed, any suitable support frame or other structure for maintaining a valve device in a body vessel can be used in embodiments in accordance with the disclosure. Furthermore, any suitable leaflet or leaflets can be used in embodiments in accordance with the disclosure. Skilled artisans will be able to select appropriate support frames, leaflets and/or other structures based on various considerations, including the nature of the body vessel within which a particular valve device is intended to be used. Examples of suitable support frames and other structures, and leaflets, can be found in the following United States patent documents, the entire contents of each of which are incorporated by reference into this disclosure in its entirety for the purpose of describing suitable components for use in valve devices in accordance with the disclosure: U.S. Pat. No. 7,524,332 to Osborne et al. for VASCULAR VALVE WITH REMOVEABLE SUPPORT COMPONENT; U.S. Pat. No. 7,520,894 to Pavcnik et al. for IMPLANTABLE VASCULAR DEVICE; U.S. Pat. No. 7,503,928 to Case et al. for ARTIFICIAL VALVE WITH CENTER LEAFLET ATTACHMENT; U.S. Pat. No. 7,402,171 to Osborne et al. for PROSTHETIC VALVE THAT PERMITS RETROGRADE FLOW; U.S. Pat. No. 7,361,189 to Case et al. for PROSTHETIC VALVE WITH PORES; U.S. Pat. No. 6,974,474 to Pavcnik et al. for MULTIPLE-SIDED INTRALUMINAL MEDICAL DEVICE; Patent Application Publication no. 20090132037 to Hoffman et al. for VALVE FRAME; Patent Application Publication no. 20090105813 to Chambers et al. for IMPLANTABLE VALVE DEVICE; Patent Application Publication no. 20070260327 to Case et al. for ARTIFICIAL VALVE PROSTHESIS WITH IMPROVED FLOW DYNAMICS; Patent Application Publication no. 20060265053 to Hunt for PROSTHETIC VALVE DEVICES AND METHODS OF MAKING AND USING SUCH DEVICES; and Patent Application Publication no. 20060235511 to Osborne for WOVEN IMPLANTABLE DEVICE.

FIG. 15 illustrates an exemplary method of making a valve device. The exemplary method 500 comprises the steps of determining 502 a desired closing pressure (Pc); determining 504 a desired opening pressure (Po), selecting 506 one or more valve leaflets; selecting 508 an appropriate means for biasing the leaflet in an open configuration based on the determined closing (Pc) and opening (Po) pressures; and attaching 510 the one or more valve leaflets to the means for biasing the leaflet in an open configuration. An optional step of attaching the one or more valve leaflets to a support frame or other means for maintaining an axial position of the leaflets in a body vessel can be included in the method. This optional step can be conducted before, concurrently with, or after the step of attaching 510 the one or more valve leaflets to the means for biasing the leaflet in an open configuration.

The inventors have determined that, for valve devices intended to be used as supplemental or replacement venous valves, a Po value of between about 1 and about 2 mmHg is suitable. The inventors believe that a Po value of less than 1 mmHg will be particularly suitable for such valve devices. Also, the inventors have determined that a Pc value of between about 10 and about 12 mmHg is suitable for such valve devices. The inventors believe that a Pc value of about 11 mmHg will be particularly suitable for such valve devices.

An optional step comprises determining a desired resting height for the valve device based on an expected or actual diameter of a body vessel in which the valve device is intended to be implanted. In exemplary methods, a resting height that is less than the expected or actual diameter of the body vessel is determined. In exemplary methods, a resting height of between about 0.1% and about 75% of the expected or actual diameter of the vessel into which they are intended to be implanted is determined. In exemplary methods, a resting height of less than about 75% of the expected or actual diameter of the vessel into which they are intended to be implanted is determined. In exemplary methods, a resting height of less than about 50% of the expected or actual diameter of the vessel into which they are intended to be implanted is determined. In exemplary methods, a resting height of less than about 25% of the expected or actual diameter of the vessel into which they are intended to be implanted is determined. In exemplary methods, a resting height of less than about 10% of the expected or actual diameter of the vessel into which they are intended to be implanted is determined.

In one exemplary method, the step of selecting an appropriate means for biasing the leaflet in an open configuration comprises selecting an appropriate spine. An optional step in this exemplary method comprises configuring the spine to have a length that is greater than an expected diameter of a body vessel in which the valve device is intended to be implanted.

The foregoing detailed description provides exemplary embodiments of the invention and includes the best mode for practicing the invention. The description and illustration of embodiments is intended only to provide examples of the invention and not to limit the scope of the invention, or its protection, in any manner.

What is claimed is:

1. An implantable valve device for regulating fluid flow through a body vessel having a vessel wall and an inner diameter, said implantable valve device comprising:
a valve leaflet having proximal and distal surfaces and adapted to move between an open configuration that permits said fluid flow through said body vessel and a closed configuration that substantially prevents fluid flow through said body vessel; and
a spine attached to the valve leaflet and biased to maintain the valve leaflet in the open configuration absent application of sufficient force on one of the proximal and distal surfaces, the spine comprising a main body attached to the leaflet, an anchor portion adapted to engage a portion of said vessel wall, and a hinge disposed between the anchor portion and the main body and adapted to enable the spine to move the leaflet between the open and closed configurations.

2. The implantable valve device according to claim 1, wherein the spine is formed of a shape memory material.

3. The implantable valve device according to claim 2, wherein the shape memory material comprises nitinol.

4. The implantable valve device according to claim 1, wherein the main body has a lengthwise axis, a first end disposed adjacent the hinge and a second end disposed opposite the first end, the first end having a first width and the second end having a second, different width;
wherein the first and second widths are measured on an axis transverse to the lengthwise axis.

5. The implantable valve device according to claim 4, wherein the first width is greater than the second, different width.

6. The implantable valve device according to claim 1, wherein the main body has a length that is greater than said inner diameter of said body vessel.

7. The implantable valve device according to claim 1, wherein said valve device has a resting height that is less than said inner diameter of said body vessel.

8. The implantable valve device according to claim 1, wherein the main body comprises a base and at least two body extensions projecting outward from the base.

9. The implantable valve device according to claim 1, wherein the main body is attached to one of the proximal and distal surfaces of the valve leaflet.

10. The implantable valve device according to claim 1, wherein the main body is embedded in the valve leaflet.

11. The implantable valve device according to claim 1, wherein a portion of the main body has a first thickness and a portion of the hinge has a second, different thickness.

12. The implantable valve device according to claim 11, wherein the second, different thickness is less than the first thickness.

13. The implantable valve device according to claim 1, wherein the spine defines an opening in the hinge.

14. The implantable valve device according to claim 1, wherein the spine defines at least two openings in the hinge.

15. The implantable valve device according to claim 1, wherein the valve leaflet comprises an extracellular matrix material.

16. The implantable valve device according to claim 1, wherein the valve leaflet comprises a tissue section.

17. The implantable valve device according to claim 1, wherein the valve leaflet comprises a polymeric material.

18. The implantable valve device according to claim 1, further comprising a support frame;
wherein the anchor portion is attached to the support frame.

19. An implantable valve device for regulating fluid flow through a body vessel having a vessel wall and an inner diameter, said implantable valve device comprising:
a valve leaflet having proximal and distal surfaces and adapted to move between an open configuration that permits said fluid flow through said body vessel and a closed configuration that substantially prevents fluid flow through said body vessel; and
a spine attached to the valve leaflet and biased to maintain the valve leaflet in the open configuration absent application of sufficient force on one of the proximal and distal surfaces, the spine comprising a main body attached to the leaflet, an anchor portion adapted to engage a portion of said vessel wall, and a hinge disposed between the anchor portion and the main body and adapted to enable the spine to move the leaflet between the open and closed configurations, the main body having a lengthwise axis, a first end disposed adjacent the hinge and a second end disposed opposite the first end, the first end having a first width and the second end having a second, different width;
wherein the first and second widths are measured on an axis transverse to the lengthwise axis; and
wherein said valve device has a resting height that is less than said inner diameter of said body vessel.

20. An implantable valve device for regulating fluid flow through a body vessel having a vessel wall and an inner diameter, said implantable valve device comprising:
a valve leaflet having proximal and distal surfaces and adapted to move between an open configuration that permits said fluid flow through said body vessel and a closed configuration that substantially prevents fluid flow through said body vessel; and
a spine attached to the valve leaflet and biased to maintain the valve leaflet in the open configuration absent application of sufficient force on one of the proximal and distal surfaces, the spine comprising a main body attached to the leaflet, an anchor portion adapted to engage a portion of said vessel wall, and a hinge disposed between the anchor portion and the main body and adapted to enable the spine to move the leaflet between the open and closed configurations, the main body comprising a base and at least two body extensions projecting outward from the base;
wherein said valve device has a resting height that is less than said inner diameter of said body vessel.
